(19) United States
(12) Patent Application Publication
(10) Pub. No.: US 2025/0246267 A1
(43) Pub. Date: Jul. 31, 2025

(54) METHOD AND DEVICE FOR PREDICTING SHAPE OF DNA STRUCTURE BASED ON DEEP LEARNING

(71) Applicant: SEOUL NATIONAL UNIVERSITY R&DB FOUNDATION, Seoul (KR)

(72) Inventors: DO NYUN KIM, Seoul (KR); JAE YOUNG LEE, Seoul (KR); TRUONG-QUOC CHIEN, Seoul (KR)

(21) Appl. No.: 18/687,007

(22) PCT Filed: Jan. 10, 2024
(86) PCT No.: PCT/KR2024/000450

§ 371 Date: Feb. 27, 2024

(30) Foreign Application Priority Data
May 19, 2023 (KR) 10-2023-0064976

(51) Int. Cl. G16B 40/30; G16B 15/20

(52) U.S. Cl. CPC G16B 40/30; G16B 15/20

(57) ABSTRACT

In a method for predicting a shape of a DNA structure based on deep learning, a DNA structure graph having nodes and edges based on a DNA structure design is acquired. A DNA structure shape corresponding to the DNA structure design based on the DNA structure graph and a DNA structure shape prediction model is output.

### CROSS REFERENCE TO RELATED APPLICATIONS AND CLAIM OF PRIORITY

This application claims benefit under 35 U.S.C. 119, 120, 121, or 365 (c), and is a National Stage entry from International Application No. PCT/KR2024/000450 filed on Jan. 10, 2024, which claims priority to the benefit of Korean Patent Application No. 10-2023-0064976 filed on May 19, 2023 in the Korean Intellectual Property Office, the entire contents of which are incorporated herein by reference.

### BACKGOUND

### 1. Technical Field

The present invention relates to a method and a device for predicting a shape of a DNA structure based on deep learning, and more specifically, to a method and a device for training a neural network model based on a hybrid training algorithm and predicting the shape of a DNA structure based on the same.

### 2. Background Art

Artificial intelligence and deep learning techniques are significantly changing the paradigm in analysis and design of molecular structures. Deep learning is a technique that performs training using a huge amount of data, and when new data are input, selects an answer with the highest probability based on training results.

Since the deep learning enables to operate adaptively according to images, and automatically finds characteristic factors during training a model based on the data, attempts to utilize this technique in the field of artificial intelligence have been increased recently.

AlphaFold and RoseTTAFold are deep learning models for predicting the sequence, structure, and function of proteins, and accelerates the design process of new proteins, such that researches for applying them to accelerating the design process of DNA structures are being conducted.

However, the DNA structure is made at the nanoscale by synthesizing tens to hundreds of complementary DNA strands, thereby having a resolution of several nanometers, and unlike the proteins, this DNA structure (particularly, a DNA origami structure) may have a complex shape, as well as the number of base pairs complementarily bound to each other is very large in a unit of thousands to tens of thousands. Thus, there is a problem in that the prediction accuracy for the shape thereof is significantly reduced with data-driven training used by existing deep learning models such as the AlphaFold and RoseTTAFold.

### SUMMARY

An object of the present invention is to provide a model for predicting a shape of a DNA structure (“DNA structure shape prediction model”), which is trained by a hybrid training algorithm based on a loss function according to data-driven loss and physics-informed loss, and an electronic device including the same.

Another object of the present invention is to provide a method and a device for predicting a shape of a DNA structure corresponding to a DNA structure design using the trained DNA structure shape prediction model.

To achieve the above objects, according to an aspect of the present invention, there is provided a method for predicting a shape of a DNA structure based on deep learning, the method including: (a) acquiring a DNA structure graph consisting of nodes and edges based on a DNA structure design; and (b) outputting a DNA structure shape corresponding to the DNA structure design based on the DNA structure graph and a DNA structure shape prediction model.

Here, the DNA structure shape prediction model may include two or more DNA structure shape prediction models, and the step (b) may include: acquiring two or more preliminary DNA structure shapes with respect to an input of the DNA structure graph for each of the two or more DNA structure shape prediction models; and outputting a preliminary DNA structure shape with the lowest energy of the two or more preliminary DNA structure shapes as the DNA structure shape.

Here, the DNA structure shape prediction model may be generated by the steps of: (c) acquiring a plurality of training DNA structure designs, and generating training DNA structure graphs corresponding to each of the acquired plurality of DNA structure designs; (d) acquiring a correct answer DNA structure shape corresponding to the plurality of training DNA structure designs based on a structural simulation program; and (e) causing a pre-stored DNA structure shape prediction model to perform training based on the plurality of training DNA structure graphs and the correct answer DNA structure.

Here, the step (c) may include: (f) acquiring training augmentation designs by performing data augmentation based on insertion of at least one base pair or deletion of at least one base pair on at least some of the plurality of acquired training DNA structure designs; and (g) generating training augmentation structure graphs corresponding to each of the training augmentation structure designs, wherein the step (e) may cause the pre-stored DNA structure shape prediction model to perform training based on the plurality of training DNA structure graphs, the training augmentation structure graphs, and the correct answer DNA structure.

Here, the step (e) may cause the pre-stored DNA structure shape prediction model to perform training by applying different loss functions to learning of the training DNA structure graphs and learning of the training augmentation structure graphs.

Here, the step (e) may cause the pre-stored DNA structure shape prediction model to perform training on the training DNA structure graphs by applying a first loss function based on a data-driven loss and a physics-informed loss.

Here, the step (e) may cause the pre-stored DNA structure shape prediction model to perform training on the training augmentation structure graphs by applying a second loss function based on a physics-informed loss.

Here, the DNA structure shape prediction model may include: a first DNA structure shape prediction model trained using the plurality of training DNA structure designs as one class; and two or more second DNA structure shape prediction models trained using the first DNA structure shape prediction model for each of two or more detailed classes, and each of the two or more detailed classes may have the plurality of training DNA structure designs classified and included according to the two or more detailed classes.

According to another aspect of the present invention, there is provided a device for predicting a shape of a DNA structure based on deep learning, the device including: a storage unit in which a DNA structure shape prediction model is stored; a graph generation unit configured to acquire a DNA structure graph consisting of nodes and edges based on a DNA structure design; and a structure prediction unit configured to output a DNA structure shape corresponding to the DNA structure design based on the DNA structure graph and a pre-trained DNA structure shape prediction model.

Here, the DNA structure shape prediction model may include two or more DNA structure shape prediction models, the structure prediction unit may be configured to: acquire two or more preliminary DNA structure shapes with respect to an input of the DNA structure graph for each of the two or more DNA structure shape prediction models included in the DNA structure shape prediction model; and determine a preliminary DNA structure shape with the lowest energy of the two or more preliminary DNA structure shapes as the DNA structure shape.

Here, the device for predicting a shape of a DNA structure based on deep learning may further include: a design acquisition unit; a correct answer shape acquisition unit; and a learning processing wherein the DNA structure shape prediction model may be generated by the processes of: acquiring, by the design acquisition unit, a plurality of training DNA structure designs; generating, by the graph acquisition unit, training DNA structure graphs corresponding to each of the acquired plurality of DNA structure designs; acquiring, by the correct answer shape acquisition unit, a correct answer DNA structure shape corresponding to the plurality of training DNA structure designs based on a structural simulation program; and causing, by the learning processing unit, the pre-stored DNA structure shape prediction model to perform training based on the plurality of training DNA structure graphs and the correct answer DNA structure.

Here, the design acquisition unit may acquire training augmentation designs by performing data augmentation based on insertion of at least one base pair or deletion of at least one base pair on at least some of the plurality of acquired training DNA structure designs, the graph acquisition unit ma y generate training augmentation structure graphs corresponding to each of the training augmentation structure designs, and the learning processing unit may cause the pre-stored DNA structure shape prediction model to perform training based on the plurality of training DNA structure graphs, the training augmentation structure graphs, and the correct answer DNA structure.

Here, the learning processing unit may cause the pre-stored DNA structure shape prediction model to perform training by applying different loss functions to learning of the training DNA structure graphs and learning of the training augmentation structure graphs.

Here, the learning processing unit may cause the pre-stored DNA structure shape prediction model to perform training on the training DNA structure graphs by applying a first loss function based on a data-driven loss and a physics-informed loss.

Here, the learning processing unit may cause the pre-stored DNA structure shape prediction model to perform training on the training augmentation structure graphs by applying a second loss function based on a physics-informed loss.

Here, the learning processing unit may generate the DNA structure shape prediction model by including: a first DNA structure shape prediction model trained using the plurality of training DNA structure designs as one class; and two or more second DNA structure shape prediction models trained using the first DNA structure shape prediction model for each of two or more detailed classes, and each of the two or more detailed classes may have the plurality of training DNA structure designs classified and included according to the two or more detailed classes.

According to various embodiments of the present invention, a DNA structure shape prediction model trained by a hybrid training algorithm based on the loss function according to data-driven loss and physics-informed loss and a device including the same may be provided, thus to predict quickly and accurately the shape of the DNA origami structure.

According to various embodiments of the present invention, an ensemble model including a plurality of shape prediction models classified and trained according to the DNA structure and a device including the same may be provided, thus to predict the shape of the monomeric DNA origami structure with high precision in almost real time.

According to various embodiments of the present invention, an ensemble model based on a hybrid training algorithm in which a data-driven training method and a physics-informed training method are combined and a device including the same may be provided, thus to predict quickly and accurately a DNA structure having a complex shape in almost real time, and predict accurately the shapes of bio, nano, and macro structures at various scales, where they lack data.

### BRIEF DESCRIPTION OF THE DRAWINGS

FIG. 1 is a conceptual diagram schematically illustrating a configuration of a device according to an embodiment of the present invention.

FIG. 2 is a block diagram illustrating configurations of a storage unit in which a shape prediction model is stored and a processing unit which performs functions based on the shape prediction model, by classifying them depending on the functions thereof in the device according to an embodiment of the present invention.

FIG. 3 is a conceptual diagram schematically illustrating an operation of generating a DNA structure graph based on DNA structure design in the device according to an embodiment of the present invention.

FIG. 4 is a conceptual diagram illustrating the schematic configuration and operation of the shape prediction model in the device according to an embodiment of the present invention.

FIG. 5 is a conceptual diagram schematically illustrating an operation of training the shape prediction model based on a hybrid training algorithm in the device according to an embodiment of the present invention.

FIG. 6 is a diagram illustrating a shape of the DNA structure (“DNA structure shape”) predicted by a shape prediction model trained in a different manner from the shape prediction model trained according to the hybrid training algorithm in the device according to an embodiment of the present invention.

FIG. 7 is a graph illustrating shape scores of the DNA structure shapes predicted by the shape prediction model trained in a different manner from the shape prediction model trained according to the hybrid training algorithm in the device according to an embodiment of the present invention.

FIG. 8 is a graph illustrating energy levels of the DNA structure shapes predicted by the shape prediction model trained in a different manner from the shape prediction model trained according to the hybrid training algorithm in the device according to an embodiment of the present invention.

FIG. 9 is a diagram illustrating a schematic configuration of an ensemble model generated based on the shape prediction models trained for each of a plurality of classes in the device according to an embodiment of the present invention.

FIG. 10 is a diagram illustrating comparison of the structure shapes predicted by the ensemble model and SNUPI corresponding to a supramolecular assembly in the device according to an embodiment of the present invention.

FIG. 11 is a graph illustrating shape scores of the structure shapes predicted by the ensemble model and SNUPI corresponding to the supramolecular assembly in the device according to an embodiment of the present invention.

FIG. 12 is a graph illustrating energy levels of the structure shapes predicted by the ensemble model and SNUPI corresponding to the supramolecular assembly in the device according to an embodiment of the present invention.

FIG. 13 is a conceptual diagram schematically illustrating a configuration of the ensemble model having an unsupervised self-refinement function in the device according to an embodiment of the present invention.

FIG. 14 is a diagram illustrating the structure shapes predicted by the ensemble model including the unsupervised self-refinement function corresponding to the supramolecular assembly in the device according to an embodiment of the present invention.

FIG. 15 is a graph illustrating energy levels of the structure shapes predicted by the ensemble model including the unsupervised self-refinement function corresponding to the supramolecular assembly in the device according to an embodiment of the present invention.

FIG. 16 is a flow chart illustrating procedures of an operation for predicting the structure shape using the shape prediction model in the device according to an embodiment of the present invention.

FIG. 17 is a flow chart illustrating procedures of an operation for training the shape prediction model configured to predict the structure shape in the device according to an embodiment of the present invention.

### DETAILED DESCRIPTION

Hereinafter, embodiments will be described in detail with reference to the accompanying drawings. However, since various changes can be made in the embodiments, the scope of the patent invention is not limited or restricted by these embodiments. It should be understood that all modifications, equivalents, and alternatives for the embodiments are included in the scope of the present invention.

The terms used in the embodiments are used only for the purpose of describing the invention, and should not be interpreted as limiting. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements, and/or components, but do not preclude the presence or addition of one or more other features, integers, steps, operations, elements, components, and/or combinations thereof.

Unless otherwise defined, all terms including technical or scientific terms used herein have the same meaning as commonly understood by one of ordinary skill in the art to which the present invention pertains. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Further, in describing the embodiments with reference to the accompanying drawings, the same reference numerals are denoted to the same components regardless of the number of the drawings, and the same configuration will not be repeatedly described. Further, in description of the embodiments, the publicly known techniques related to the present invention, which are verified to be able to make the purport of the present invention unnecessarily obscure, will not be described in detail.

In addition, in describing components of the embodiment, the terms such as first, second, A, B, (a), (b), and the like may be used. These terms are intended to divide the components from other components, and do not limit the nature, sequence or order of the components.

It will be understood that when a component is described to as being “connected,” “combined” or “coupled” to another component, the component may be directly connected or coupled the another component, but it may be “connected,” “combined” or “coupled” to the another component intervening another component may be present.

In addition, it will be understood that when a component is described as being “connected” or “combined” by communication to another component, that component may be connected or combined by wireless or wired communication to the another component, but it may be “connected” or “combined” to the another component intervening another component may be present.

Components included in one embodiment and components including common functions will be described using the same names in other embodiments. The description given in one embodiment may be applied to other embodiments, and therefore will not be described in detail within the overlapping range, unless there is a description opposite thereto.

The device and/or ‘data’ processed by the device may be expressed in terms of the ‘information’. Here, information may be used as a concept including the data.

The present invention relates to a method and a device for predicting a shape of a DNA structure based on deep learning. To describe in more detail, the present invention provides a method for predicting a multidimensional (e.g., two-dimensional, three-dimensional, etc.) shape of the DNA structure corresponding to a DNA structure design on the basis of a DNA structure shape prediction model based on deep learning, a method for training the DNA structure shape prediction model, and a device for performing the methods.

Hereinafter, preferred embodiments of the present invention will be described with reference to the drawings. The drawings attached to this specification serve to further understand the technical idea of the present invention along with the description detailed of the invention, and therefore, the present invention should not be construed as limited to the matters described in such drawings.

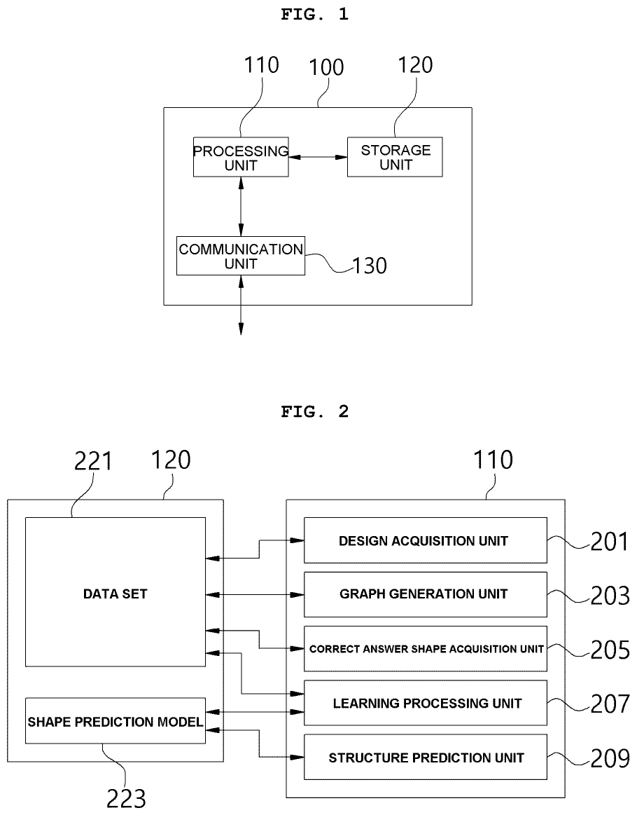

Hereinafter, a configuration of a device will be schematically described with respect to FIG. 1.

FIG. 1 is a conceptual diagram schematically illustrating the configuration of the device according to an embodiment of the present invention.

Referring to FIG. 1, a device 100 for predicting a shape of a DNA structure may include a processing unit 110, a storage unit 120, and a communication unit 130.

The processing unit 110 includes at least one processor (or controller), and may process control commands related to operations performed by the device 100 through at least one program (app, application, tool, plug-in, software and the like).

For example, the processing unit 110 may cause a shape prediction model to perform training through a shape prediction program, and predict the DNA structure shape corresponding to the acquired DNA structure design. In this case, the shape prediction program may be stored in the storage unit 120 of the device 100 and/or a storage unit of another device connected to the device 100.

According to various embodiments of the present invention, the shape prediction program may include at least one neural network model based on deep learning. To describe in more detail, the shape prediction program may include a shape prediction model (or a DNA structure shape prediction model) based on a graph neural network (GNN).

In this case, the shape prediction model may be a neural network model (e.g., a DNA origami graph neural network model) configured to predict the DNA structure shape corresponding to the DNA structure design in a DNA paper-folding (or origami) method.

The shape prediction program may include at least some of programs (and/or algorithms) for performing operations of: acquiring a DNA structure design; generating a DNA structure graph based on the DNA structure design; causing the shape prediction model to perform training based on the DNA structure graph; and predicting the DNA structure shape based on the DNA structure graph, etc., which will be described through the embodiments of the present invention.

The processing unit 110 may share data processing and/or processing results with at least one device (e.g., a user device) connected to the device 100 through the shape prediction model.

Hereinafter, in various embodiments, it may be understood that performing the operations according to the control commands by the device 100 indicates performing the at least one program predetermined operations through related to at least one control command processing of the device 100 and/or the shape prediction program.

Here, it will be described that the control command processing is performed through the at least one program or the shape prediction program installed in the device 100, but it is not limited thereto, and may be performed through another program or a temporary installation program previously provided and installed in the storage unit 120.

According to one embodiment, the control command processing may be performed by the processing unit 110 based on at least a portion of a database provided free of charge or for a fee in an external device connected to the device 100.

The operation of the device 100 is performed based on data processing and device control of the processing unit 110, and the processing unit 110 may also perform the predetermined functions on the basis of the control commands received through an input/output unit (not shown) and/or the communication unit of the device 100.

In addition, in processing data acquired through the communication unit 130, the processing unit 110 may process the data based on an identified user. For example, the processing unit 110 may identify a user based on user information received from the user device connected thereto through the communication unit 130 and/or user information acquired through an input unit (or input/output unit) connected to the device 100, and perform an operation according to the control command input by the identified user.

The storage unit 120 may store various data processed by at least one component (e.g., the processing unit 110 or the communication unit 130) of the device 100. The data may include, for example, a program for control command processing or data processed through the program, and/or input data and output data related thereto.

The storage unit 120 may include an artificial algorithm for control command processing, which includes at least some of an artificial neural network algorithm, a blockchain algorithm, a deep learning algorithm, and a regression analysis algorithm based on at least some of the mechanisms, operators, language models, and big data related thereto.

The shape prediction program or shape prediction model stored in the storage unit 120 may include at least some of the above-described artificial intelligence algorithms.

The shape prediction program includes at least some of artificial intelligence algorithms for generating a DNA structure graph consisting of nodes and edges based on the acquired DNA structure design, and/or may be connected to an artificial intelligence algorithm for the same.

In addition, the artificial intelligence algorithm that forms the shape prediction program may be formed by classifying it into a plurality of neural network models.

For example, the artificial intelligence algorithm for the shape prediction program may include an algorithm based on an attention mechanism of a transformer layer. More preferably, the artificial intelligence algorithm for the shape prediction program may include an algorithm based on a multi-head attention mechanism.

Hereinafter, in describing various embodiments of the present invention, the operation of the shape prediction model should be understood as the operation of the shape prediction model or an operation of the shape prediction program including the shape prediction model.

The storage unit 120 may include at least one DNA structure design for use in predicting the DNA structure shape.

To describe in more detail, the storage unit 120 may store at least some of training DNA structure designs and correct answer DNA structure shapes, which are provided for training the shape prediction model. In this case, the training DNA structure designs and the correct answer DNA structure shapes may be stored as a data set for training the shape prediction model.

The storage unit 120 may include data for confirming and processing the predetermined control and operations through signals received from each of devices included in the input/output unit (not shown) and/or through the communication unit 130.

The operations performed in relation to the storage unit 120 are processed by the processing unit 110, and data for processing the related operations, data in process, processed data, preset data, and the like may be stored in the storage unit 120 as a database.

The data stored in the storage unit 120 may be changed, modified, deleted, and/or generated as new data by the processing unit 110 based on user input of the identified user.

The storage unit 120 may store device setting information of the device 100. The device setting information may be setting information on the device 100 and at least some of functions and services provided by the device 100.

The storage unit 120 may store user information (or user account) for at least one user. As the user information, at least some piece of information among user identification information (e.g., identification, ID), password, and user-customized setting information may be stored. The user-customized setting information is setting information on at least some of control authority and/or the functions of the device 100, and may be set and stored according to the administrator input.

Here, at least some piece of the user information may be stored as data of the shape prediction program.

The storage unit 120 may include a volatile memory, a non-volatile memory, and/or a computer-readable recording medium as known in the art. In this case, the computer-readable recording medium may store a computer program for performing the operations according to an embodiment of the present invention by the device 100 based on various embodiments of the present invention.

The communication unit 130 may support establishment of a wired communication channel or establishment of a wireless communication channel between the device 100 and at least one other device (e.g., the user device or an administrator device capable of controlling the device 100), and performing communication through the established communication channel.

The communication unit 130 may perform operations such as modulation/demodulation and encryption/decryption, etc., during performing communication, which is obvious to those skilled in the art, and therefore will not be described in more detail.

The communication unit 130 is operated dependently on or independently from the processing unit 110, and may include one or more communication processors which support wireless communication and/or wired communication.

According to an embodiment, when supporting the wireless communication, the communication unit 130 may include at least some communication modules of wireless communication modules, for example, a cellular communication module, a near field communication module, and a global navigation satellite system (GNSS) communication module.

When supporting the wired communication, the communication unit 130 may include at least some communication modules of wired communication modules, for example, a local area network (LAN) communication module, a power line communication module, a controller area network (CAN) communication module, and a serial peripheral interface (SPI) communication module.

To describe in more detail, the communication unit 130 may communicate with the external device by wired and/or wirelessly through near field communication networks such as Bluetooth, Bluetooth Low Energy (BLE), WiFi, WiFi direct, infrared data association (IrDA), ZigBee, UWB, and radio frequency (RF), and/or far field communication networks such as a cellular network, the Internet or a computer network (e.g., LAN or WAN).

Various types of communication modules that form the communication unit 130 may be integrated into one component (e.g., a single chip), or may be implemented as a plurality of separate components (e.g., a plurality of chips).

Referring to FIG. 1, it shows that the communication unit 130 performs communication with an external device of the device 100, but it is not limited thereto, and for example, may perform communication with the storage unit 120, and/or at least some of the various components of device 100 which are not illustrated and configured in FIG. 1.

In addition, although not shown in FIG. 1, the device 100 may further include an input/output unit.

The input/output unit may include an input unit and an output unit. The input/output unit may include at least some of an input unit (not shown) configured to input data, such as a keyboard, mouse, or microphone, and an output unit (not shown) configured to output data, such as a driving unit, a display unit (e.g., a display), a speaker or the like.

In this case, the display unit may be configured as a touch screen capable of touch input.

According to various embodiments of the present invention, the device 100 or the user device may include at least some of functions of all information and communication devices including a mobile communication terminal, a multimedia terminal, a wired terminal, a fixed terminal, an internet protocol (IP) terminal and the like.

The device 100 is a device for control command processing, and may include at least some functions of a workstation or a large-capacity database, or may be configured to be connected with at least some of the workstation or the large-capacity database through communication.

As the device 100 or the user device connected to device 100, a mobile phone, a personal computer (PC), a portable multimedia player (PMP), a mobile internet device (MID), a smartphone, a tablet PC, a phablet PC, a laptop computer, and the like may be exemplified.

According to various embodiments, the user device will be described as a device which is connected to the device 100 and communicates therewith. For example, although not illustrated throughout the drawings, the user device may be a smartphone of the user, which is connected to the device 100 through wireless communication and transmits the user input so that the device 100 the operations according to an embodiment of the present invention.

The user device may be connected to the device 100 through at least one program installed therein, or may be connected to the device 100 through at least one web page accessed on online by the device 100.

To this end, the device 100 may include at least some of the functions of a server and/or a terminal for providing a service for predicting a DNA structure shape.

FIG. 2 is a block diagram illustrating configurations of the storage unit in which the shape prediction model is stored and the processing unit which performs functions based on the shape prediction model, by classifying them depending on the functions thereof in the device according to an embodiment of the present invention.

According to various embodiments of the present invention, as described above, the device 100 may include the shape prediction program which performs prediction of the DNA structure shape and trains the shape prediction model for prediction of the DNA structure shape.

According to various embodiments of the present invention, the shape prediction program of the device 100 may include a shape prediction model based on the graph neural network (GNN).

That is, the device 100 may predict the DNA structure shape corresponding to the DNA structure design acquired through the pre-trained shape prediction model, and thereby a prediction accuracy of the DNA structure shape by the shape prediction program of the device 100 can be improved through training of the shape prediction model.

First, in the device 100, the storage unit 120, in which data related to the operations of the processing unit 110 for predicting the DNA structure shape using the shape prediction model or training the shape prediction model are stored as described above, may include a data set 221 and a shape prediction model 223.

In addition, in the device 100, the processing unit 110 for predicting the DNA structure shape based on the data set 221 and the shape prediction model 223 stored in the storage unit 120, or training the shape prediction model, may include at least one element of a design acquisition unit 201, a graph generation unit 203, a correct answer shape acquisition unit 205, a learning processing unit 207, and a structure prediction unit 209.

First, according to one embodiment of the data set 221 for training the shape prediction model 223, the data set 221 may include a plurality of DNA structure designs. In this case, the plurality of DNA structure designs may be classified into a training data set and a test data set at a predetermined ratio.

In this state, at least some of the DNA structure designs included in the data set 221 may be labeled. More preferably, each DNA structure design included in the data set 221 may be labeled.

In this state, DNA structure shape information, for example, three-coordinates of atoms, may be labeled for each DNA structure design included in the data set 221. Here, information may include information the structure shape based on molecular dynamics simulation.

According to various embodiments, the data set 221 may further include an augmentation structure design generated based on data augmentation. In this case, the augmentation structure design may be stored in an unlabeled state.

Here, the operation of generating an augmentation structure design may be performed through the design acquisition unit 201.

The design acquisition unit 201 may acquire training augmentation designs by performing data augmentation based on insertion of at least one base pair or deletion of at least one base pair on at least some of the DNA structure designs stored in the data set.

Here, the design acquisition unit 201 may perform insertion or deletion of the base pair on the DNA structure design so as to satisfy an energy change within a preset value.

In addition, the data set 221 may include at least one correct answer DNA structure shape corresponding to the training DNA structure designs or training augmentation structure designs.

According to one embodiment, the correct answer DNA structure shape may be generated based on a training DNA structure graph. To this end, the graph generation unit 203 may generate a DNA structure graph or an augmentation structure graph based on the DNA structure design or the augmentation structure design.

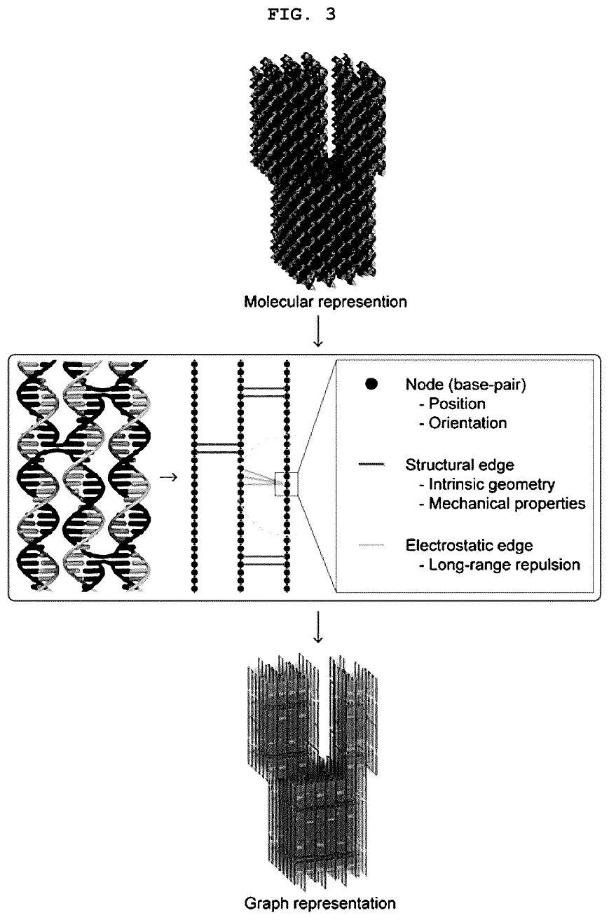

To describe in more detail, as shown in FIG. 3, the DNA structure graph processed as an input of the shape prediction model 223 may be formed as a graph including nodes and edges corresponding to the base pairs of the DNA structure design and a binding relationships between the base pairs.

FIG. 3 is a conceptual diagram schematically illustrating an operation of generating a DNA structure graph based on the DNA structure design in the device according to an embodiment of the present invention.

Referring to FIG. 3, in forming the DNA structure graph, the graph generation unit 203 may model the base pairs included in the DNA structure design, for example, the training DNA structure design of the data set 221 with nodes having information on the position and orientation.

According to one embodiment, the number of base pairs that form the training DNA structure may be 1,000 to 17,000. However, it is not limited thereto, and the number of base pairs that form the training DNA structure may be designed in various ways.

Based on the binding relationship between the base pairs included in the DNA structure design, the graph generation unit 203 may model a structural edge representing a covalent bond between the base pairs and an electromagnetic edge representing an electrostatic interaction due to a negative charge of DNA.

Here, the structural edge includes intrinsic geometry information and mechanical property information according to the base, and the electromagnetic edge may be configured to include information on a repulsion force (or a distance function for the repulsion force) formed between the base pairs.

The graph generation unit 203 may generate a DNA structure graph corresponding to the DNA structure design based on modeling of the nodes and edges.

As described above, the graph generation unit 203 may generate a DNA structure graph corresponding to the DNA structure design, may and also further generate an augmentation structure graph corresponding to the augmentation structure design.

Then, the correct answer shape acquisition unit 205 may acquire the correct answer DNA structure shape based on the generated DNA structure graph.

Here, the correct answer DNA structure shape is a DNA structure shape in three-dimensional equilibrium (or a final configuration in equilibrium, Ground-truth (GT)), may be generated based on at least one simulation program which simulates the DNA structure shape (e.g., Structured NUcleic-acids Programming Interface, SNUPI) based on a multi-scale physics-based model.

According to one embodiment, at least one program which simulates the DNA structure shape may be stored in the storage unit 120, or may be stored outside the device 100, for example, in a server.

When at least one program which simulates the DNA structure shape is stored in the server, the correct answer shape acquisition unit 205 may transmit the training DNA structure graph (or the training DNA structure design) to the server, and receive the correct answer DNA structure shape corresponding to the training DNA structure graph (or the training DNA structure design) from the server.

In addition, the correct answer shape acquisition unit 205 may classify detailed classes for the training DNA structure design or the training DNA structure graph corresponding to the training DNA structure design.

For example, the correct answer shape acquisition unit 205 may classify the detailed classes for the training DNA structure design or the training DNA structure graph based on information labeled in the training DNA structure design or the correct answer DNA structure shape.

According to one embodiment, the detailed classes may include 6 detailed classes of: a block structure, a curved structure, a twisted structure, a hinged structure, a two-dimensional wireframe structure, and a three-dimensional wireframe structure.

In addition, the correct answer shape acquisition unit 205 may set the training DNA structure designs or the training DNA structure graphs included in the data set 221 as one class (or one detailed class).

The learning processing unit 207 may cause the shape prediction model 223 to perform training based on the data set 221 configured as described above.

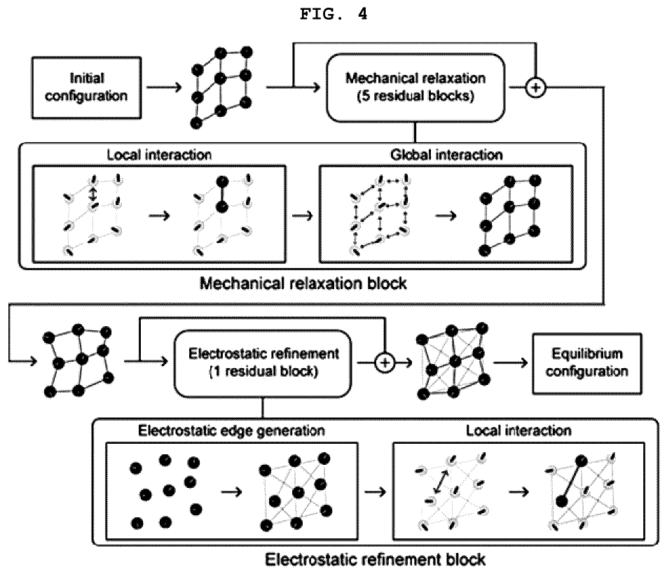

Referring to FIG. 4, the shape prediction model 223 may consist of a neural network model that predicts the DNA structure shape (the DNA structure shape or equilibrium configuration in three-dimensional equilibrium) predicted based on the DNA structure graph (or initial configuration). FIG. 4 is a conceptual diagram illustrating the schematic configuration and operation of the shape prediction model in the device according to an embodiment of the present invention.

The shape prediction model 223 that predicts the DNA structure shape using the DNA structure graph as an input may include a mechanical relaxation block, an electromagnetic refinement block, and a residual block.

Referring to FIG. 4, it shows that the shape prediction model includes five mechanical relaxation blocks and one electromagnetic refinement block, but it is not limited thereto, and the number of mechanical relaxation blocks or the number of electromagnetic refinement blocks may be variously modified.

According to one embodiment of the mechanical relaxation block, the mechanical relaxation block may be an element designed to find a structural configuration in which a mechanical strain energy is minimized based on the structure used in ResNet, or DenseNet.

According to one embodiment of the electromagnetic refinement block, it may be an element which is a structure used in a transformer model, includes a convolutional layer and an activation function, rectified linear unit (ReLU), and is designed to refine the configuration of the mechanically relaxed structure by reflecting an electrostatic repulsion.

According to one embodiment of the residual block, it may be an element which implements skip connection based on the structure used in ResNet, and is designed to solve a problem of vanishing gradient occurring in the mechanical relaxation block while taking charge of the connection between the mechanical relaxation block and the electromagnetic refinement block.

For the shape prediction model 223, the DNA structure graph (or values of the DNA structure graph) consisting of the nodes and edges may be processed as an input, and the predicted DNA structure shape may be processed as an output.

In the mechanical relaxation block, it may be configured so that the positions and orientations of the nodes are updated locally and globally in residual blocks undertaken using graph neural layers, and over-smoothing is prevented through a connection between layers.

In each of the mechanical relaxation block, the electromagnetic refinement block, and the residual block, it may be configured so that the prediction results of local displacement and rotation angle are propagated to the entire graph based on the multi-head attention mechanism of the transformer layer.

It is configured in a way that the positions and orientations of the nodes that form the structure between the residual blocks are gradually updated in gated recurrent unit cells, and in the case of a structure with relatively large deformation, it may be supported to capture long-term dependency between the layers.

As described above, configuration information of the mechanically relaxed structure may be processed as an input of the electromagnetic refinement block.

The electromagnetic refinement block may be configured to generate an electromagnetic edge between adjacent nodes within a distance of several nanometers (nm), more specifically 0 to 3 nm, and more preferably 2.5 nm. It may be configured so that the configuration of the final DNA structure shape is predicted by performing repetitive calculations between the electromagnetic edges through the electromagnetic refinement block.

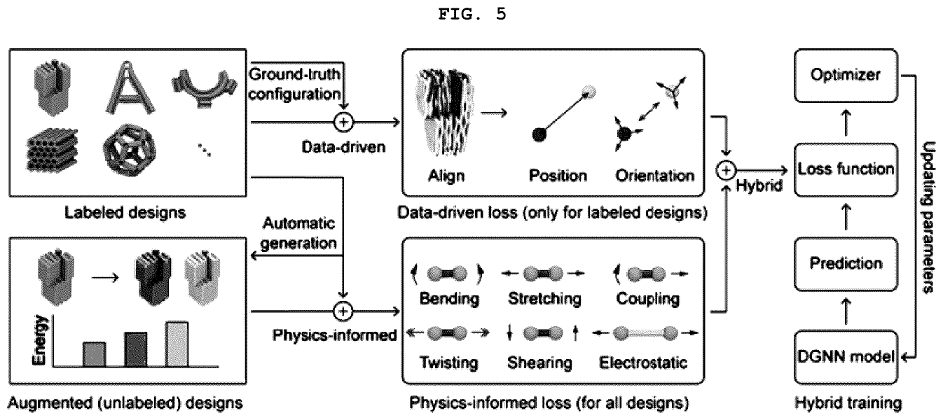

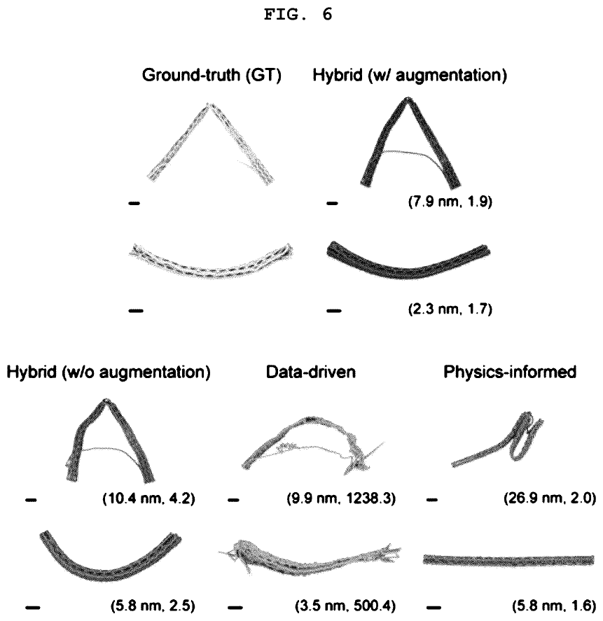

The learning processing unit 207 causes the shape prediction model 223 to be configured as described above to perform training. In this case, as shown in FIG. 5, training of the shape prediction model 223 may be performed based on a hybrid data-driven and physics-informed training algorithm (hereinafter, a hybrid training algorithm).

FIG. 5 is a conceptual diagram schematically illustrating an operation of training the shape prediction model 223 based on the hybrid data-driven and physics-informed training algorithm in the device according to an embodiment of the present invention.

The learning processing unit 207 may cause the shape prediction model 223 to perform training based on the training DNA structure graph, a training DNA augmented graph and the correct answer DNA structure shape.

In this case, the learning processing unit 207 may cause the shape prediction model 223 to perform training using a loss function on the basis of a data-based loss (e.g., data-driven loss) for the training DNA structure graph.

More preferably, the learning processing unit 207 may cause the shape prediction model 223 to perform training using a loss function on the basis of the data-driven loss and a physical information-based loss (e.g., physics-informed loss) for the training DNA structure graph.

In addition, the learning processing unit 207 may cause the shape prediction model 223 to perform training using a loss function based on the data-driven loss or the physics-informed loss for the training DNA structure graph.

Here, the loss function based on the data-driven loss (e.g., data-driven loss function) may be a function for a mean square error of the positions and orientations of the nodes between structure the DNA shape (equilibrium configuration) and the correct answer DNA structure shape which are predicted based on the shape prediction model 223.

In addition, the loss function based on the physics-informed loss (e.g., physics-informed loss function) may be a function for mechanical configuration and energy differences between the DNA structure shape (equilibrium configuration) and the correct answer DNA structure shape which are predicted based on the shape prediction model 223.

In performing training of the shape prediction model 223, the learning processing unit 207 performs training to minimize the loss function evaluated for the design (or graph) used for training. To this end, it is possible to optimize parameters of the shape prediction model 223 (or the GNN that forms the shape prediction model) by using an optimizer with a step decay learning rate scheduler (e.g., Adam, AdamW or the like).

As described above, the performance of the shape prediction model 223 trained based on the hybrid training algorithm will be described with reference to FIGS. 6 to 8.

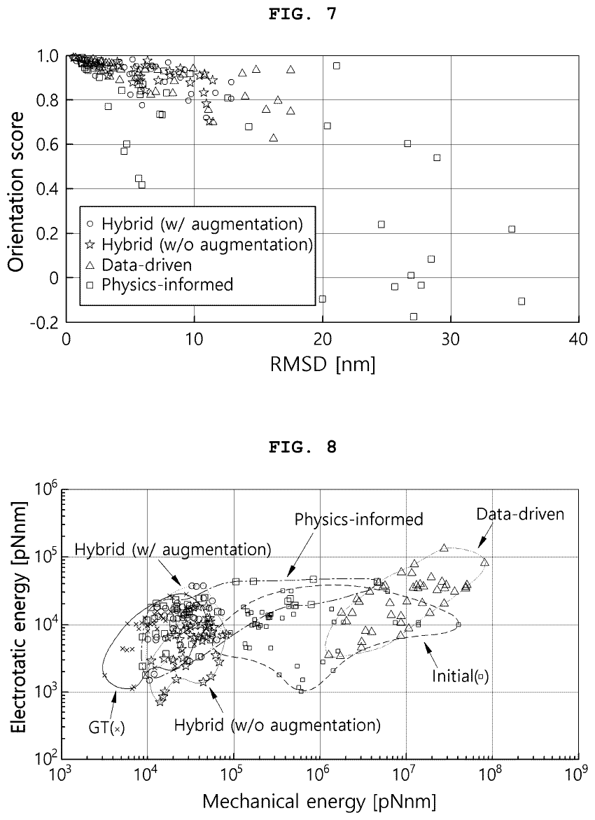

FIG. 6 is a diagram illustrating a DNA structure shape predicted by a shape prediction model trained in a different manner from the shape prediction model trained according to the hybrid training algorithm in the device according to an embodiment of the present invention. FIG. 7 is a graph illustrating shape scores of the DNA structure predicted by the shape prediction model trained in a different manner from the shape prediction model trained according to the hybrid training algorithm in the device according to an embodiment of the present invention. FIG. 8 is a graph illustrating energy levels of the DNA structure shapes predicted by the shape prediction model trained in a different manner from the shape prediction model trained according to the hybrid training algorithm in the device according to an embodiment of the present invention.

According to FIGS. 6 to 8, the shape prediction model 223 according to an embodiment of the present invention uses a model trained based on a data set consisting of a total of 300 DNA structure designs, including 252 training DNA structure designs and 48 test structure designs, and the hybrid training algorithm, and derives results by classifying a DNA structure shape (Hybrid, w/augmentation) predicted using a shape prediction model 223 (w/augmentation) on which data augmentation (w/augmentation) is performed and a DNA structure shape (Hybrid, w/o augmentation) predicted using a shape prediction model 223 (w/o augmentation) on which data augmentation (w/o augmentation) is not performed.

Here, when performing the data augmentation, the design acquisition unit 201 generated two training augmentation designs for each of 252 training DNA structure designs.

In addition, as described above, in the learning processing unit 207, both the data-driven loss and physics-informed loss were used for training in the case of the labeled design (training DNA structure design), but only the physics-informed loss was used in the training augmentation design.

The DNA structure shape compared to this includes a DNA structure shape (Data-driven) predicted using a shape prediction model trained by a data-driven approach, a DNA structure shape (Physics-informed) predicted using a shape prediction model trained by a physics-informed approach, and a DNA structure shape (GT) predicted based on SNUPI.

The DNA structure shapes illustrated through FIGS. 6 to 8 are based on the results acquired by processing 48 test DNA structure graphs as an input to the respective shape prediction models of DNA structure shape (GT), DNA structure shape (Hybrid, w/augmentation), DNA structure shape (Hybrid, w/o augmentation), DNA structure shape (Data-driven), and DNA structure shape (Physics-informed).

In FIG. 6, the values in parentheses of the respective DNA structure shape (GT), DNA structure shape (Hybrid, w/augmentation), DNA structure shape (Hybrid, w/o augmentation), DNA structure shape (Data-driven), and DNA structure shape (Physics-informed) represent a root mean square deviation (RMSD) and a ratio of total energy to the DNA structure shape (GT). Here, the scale bar is defined by 10 nm.

Here, the RMSD may represent a root mean square distance between the nodes of the DNA structure shape and the DNA structure shape (GT).

FIG. 7 is a graph illustrating shape scores, for example, values of the RMSD and OS (Orientation score) for each of the DNA structure shape (GT), DNA structure shape (Hybrid, w/augmentation), DNA structure shape (Hybrid, w/o augmentation), DNA structure shape (Data-driven), and DNA structure shape (Physics-informed).

Here, the OS may represent the orientation score defined by an inner product of the orientation vectors for each node of the DNA structure shape.

According to FIG. 7, it can be confirmed that the hybrid training algorithm of the shape prediction model 223 (w/augmentation), which predicted the DNA structure shape (Hybrid, w/augmentation), has the highest performance.

For example, it can be confirmed that the shape prediction model 223 (w/augmentation) used to predict the DNA structure shape (Hybrid, w/augmentation) predicted a DNA structure shape closest to the DNA structure shape (GT) value by predicting the DNA structure shape with the lowest RMSD and highest OS values.

FIG. 8 is a graph illustrating energy levels, for example, values of mechanical energy and electrostatic energy for each of the DNA structure shape (GT), DNA structure shape (Hybrid, w/augmentation), DNA structure shape (Hybrid, w/o augmentation), DNA structure shape (Data-driven), and DNA structure shape (Physics-informed).

According to FIG. 8, it can be confirmed that the hybrid training algorithm of the shape prediction model 223 (w/augmentation), which predicted the DNA structure shape (Hybrid, w/augmentation), shows the energy level closest to that of the DNA structure shape (GT).

On the other hand, it may not be easy to accurately predict the DNA structure shape even by a shape prediction model based on the data augmentation and hybrid training algorithm in a state where the number of designs not only whose structural features of the labeled DNA structure design (or DNA structural graph) very diverse and distinct but also whose features are similar to each other is limited.

Therefore, the learning processing unit 207 may apply an ensemble approach in which multiple shape prediction models are combined. In this case, it is possible to train a separate and independent shape prediction model for each class using the data set 221 in which the training DNA structure design (or the training DNA structure graph) is classified according to the class of the learning processing unit 207.

In more detail, as described above, when the training DNA structure design included in the data set 221 is classified into 6 detailed classes, the learning processing unit 207 may generate a total of 7 trained shape prediction models by independently training the shape prediction model 223 for each of the 6 detailed classes, and then training the shape prediction model 223 using the entire data set 221 as one class (or detailed classes).

The learning processing unit 207 may configure an ensemble shape prediction model (hereinafter, an ensemble model) including a total of seven trained shape prediction models.

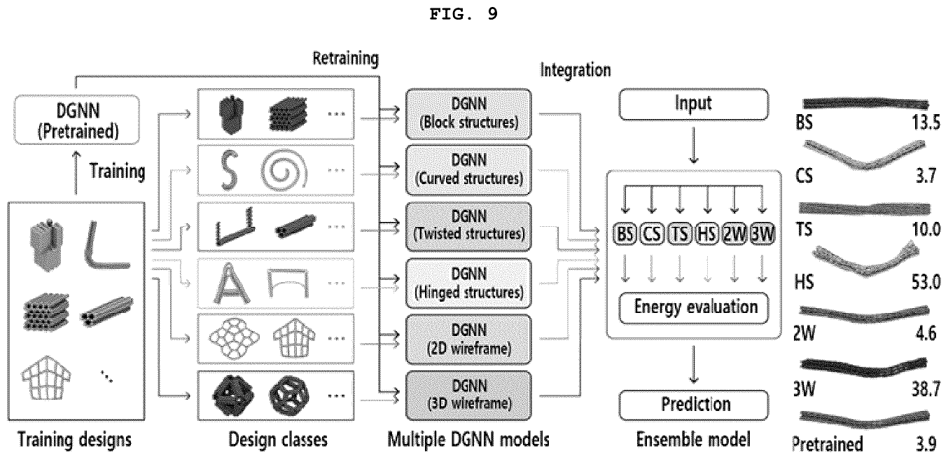

In this case, as shown in FIG. 9, the ensemble model may be configured to acquire DNA structure shapes (equilibrium configurations) predicted by each of the seven trained shape prediction models corresponding to the input initial configuration, and determine a structure shape with the lowest energy as the final DNA structure shape.

According to the above description, it has been described that the ensemble model includes seven shape prediction models, but it is not limited thereto, and the number of shape prediction models that form the ensemble model may be changed based on the number of the detailed classes.

FIG. 9 is a diagram illustrating a schematic configuration of the ensemble model generated based on the shape prediction models trained for each of a plurality of classes in the device according to an embodiment of the present invention.

In the operation of predicting the DNA structure shape for an input specific DNA structure design by the structure prediction unit 209 using the ensemble model, the DNA structure shape of a shape with the lowest energy may be output as the final result.

Referring to FIG. 9, the structure prediction unit 209 may determine and output the DNA structure shape of a CS shape with the lowest energy as the final shape among a plurality of preliminary structure shapes acquired as a result of processing the ensemble model corresponding to the specific DNA structure design.

Here, in training the plurality of shape prediction models to form the ensemble model, the learning processing unit 207 may cause each of the shape prediction models to perform training based on the hybrid training algorithm.

As described above, the learning processing unit 207 may perform prediction of a supramolecular structure, such as a polyhedron consisting of a plurality (e.g., tens to hundreds) of DNA origami monomers using the ensemble model.

At this time, in generating graph information on a hierarchically assembled large assembly, the graph generation unit 203 may generate the graph by introducing an interface edge for interaction at an interface between single structures.

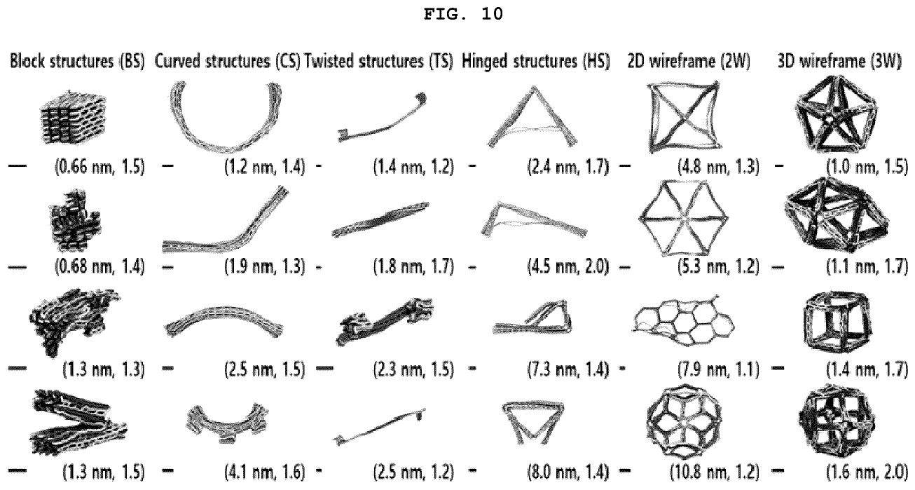

Through FIGS. 10 to 12, the single DNA structure predicted using the ensemble model according to an embodiment of the present invention may be compared with the single DNA structure (GT) predicted using SNUPI.

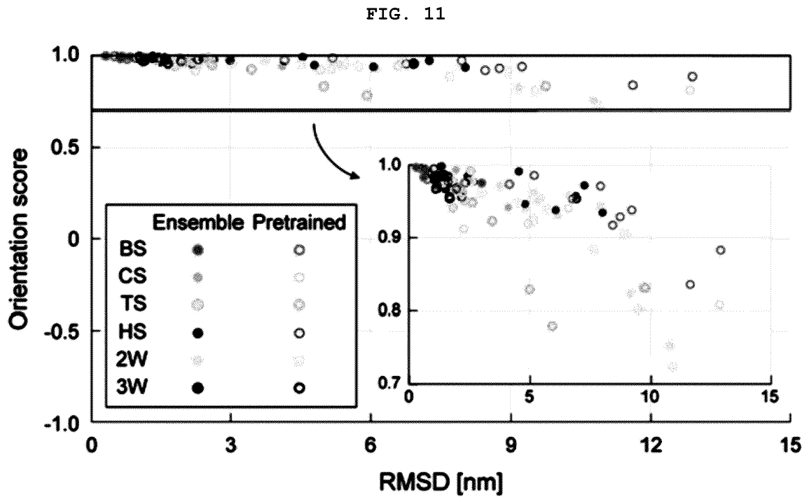

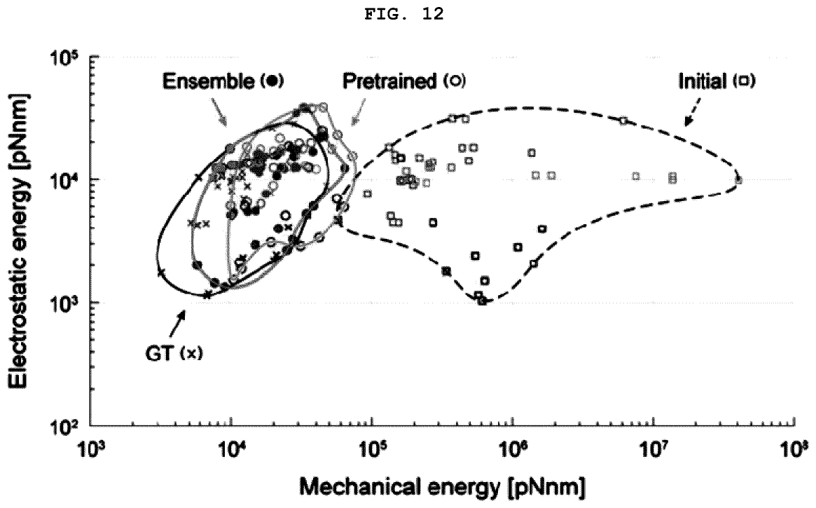

FIG. 10 is a diagram illustrating comparison of the structure shapes predicted by the ensemble model and SNUPI corresponding to a single DNA structure in the device according to an embodiment of the present invention. FIG. 11 is a graph illustrating shape scores of the structure shapes predicted by the ensemble model and SNUPI corresponding to the single DNA structure in the device according to an embodiment of the present invention. FIG. 12 is a graph illustrating energy levels of the structure shapes predicted by the ensemble model and SNUPI corresponding to the single DNA structure in the device according to an embodiment of the present invention.

Referring to FIG. 10, it can be confirmed that the single DNA structure shape (colored) predicted by the ensemble model was matched well with the shape (gray) predicted by SNUPI, and all types of deformation, including distortions of block structures, curved structures, twisted structures, hinged structures, two-wireframe structures, and three-wireframe structures, were successfully predicted.

Referring to FIG. 11, the inference time of the ensemble model for predicting the DNA structure shape based on the test DNA structure design was measured in a range of 0.55 to 1.47 seconds, with an average of 0.92 seconds, which was a prediction close to quasi-real time.

It was confirmed that the RMSD and OS values were 2.97 nm and 0.97 on average with standard deviations of 2.59 and 0.04, respectively. These results indicate that both the overall DNA structure shape and the local DNA composition can be accurately estimated from the structure information input using the ensemble model.

In addition, as shown in FIG. 12, it can be confirmed that the energy level predicted by the ensemble model is very close to the energy level of the GT structure. A ratio of the total energy of the initial configuration to that of the GT structure was 103.5 on average and in the case of the predicted equilibrium configuration, it was decreased significantly to 1.4.

This significant decrease in the energy was observed for all types of deformations in the block structure, curved structure, twisted structure, hinged structure, two-dimensional wireframe structure, and three-dimensional wireframe structure. In summary, the ensemble model according to the embodiment of the present invention may provide more accurate predictions of shape and energy level than the pre-trained model, especially the highly deformed structure.

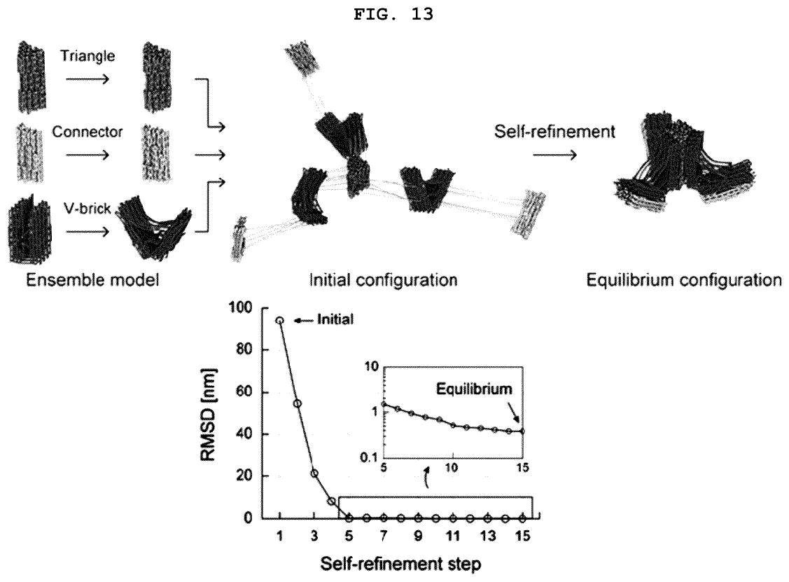

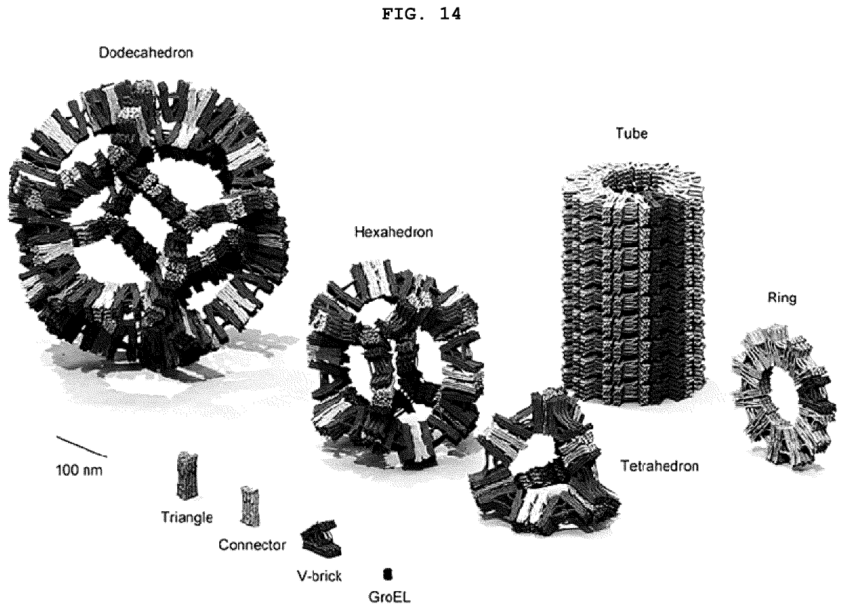

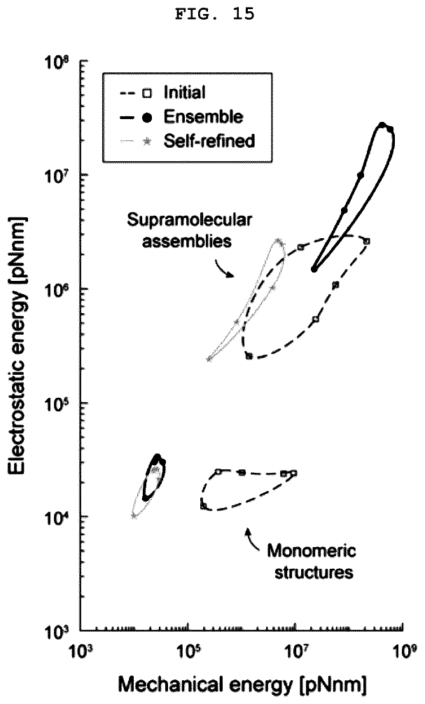

In addition, the ensemble model may be configured to perform training of the ensemble model or the shape prediction models included therein based on the unsupervised learning function of a GNN model. For example, the ensemble model may be configured to evaluate physical information of the graph based on the GNN model, which will be described with reference to FIGS. 13 to 15. FIG. 13 is a conceptual diagram schematically illustrating the configuration of the ensemble model having an unsupervised self-refinement function in the device according to an embodiment of the present invention. FIG. 14 is a diagram illustrating the structure shapes predicted by the ensemble model including the unsupervised self-refinement function corresponding to the supramolecular assembly in the device according to an embodiment of the present invention. FIG. 15 is a graph illustrating energy levels of the structure shapes predicted by the ensemble model including the unsupervised self-refinement function corresponding to the supramolecular assembly in the device according to an embodiment of the present invention.

Referring to FIG. 13, the initial configuration for the supramolecular assembly having a tripod structure was generated using multiple DNA structure shapes predicted by the ensemble model, and the ensemble model may be configured to find a configuration with the minimized energy among various deformations of the initial configuration.

According to one embodiment, in predicting the supramolecular structure shape corresponding to the supramolecular assembly, the ensemble model may be configured to predict DNA structure shapes corresponding to the DNA structure designs that form the supramolecular assembly, and determine a configuration with the minimized energy of the predicted DNA structure shapes as the supramolecular structure.

As described above, the ensemble model may be trained to exclude the data-driven loss of the operation for predicting the supramolecular structure shape and minimize the energy through the operation of predicting the DNA structure shape.

That is, this unsupervised self-refinement function may be configured to perform training of the parameters of the ensemble model so as to find the energy-minimizing configuration based on the structure shapes predicted by the ensemble model.

FIG. 14 shows tens to hundreds of DNA origami structures (V-brick, connector, triangle structures) predicted through an ensemble model including self-refinement function, and macrostructures which connect them. Radii of the predicted polyhedra (Tetrahedron: 131 nm, Hexahedron: 151 nm, Dodecahedron: 217 nm) were matched well with the experimental raddi (Tetrahedron: 125 nm, Hexahedron: 150 nm, Dodecahedron: 210 nm). Protein GroEL is illustrated for size comparison.

Referring to FIG. 15, it can be confirmed that the ensemble model including the unsupervised self-refinement function may predict the exact shape (or geometry) of the supramolecular structure by reducing the total energy of the supramolecular assembly.

Hereinafter, procedures of an operation for predicting the DNA structure shape using the ensemble model and an operation for training the ensemble model in the device according to an embodiment of the present invention will be described.

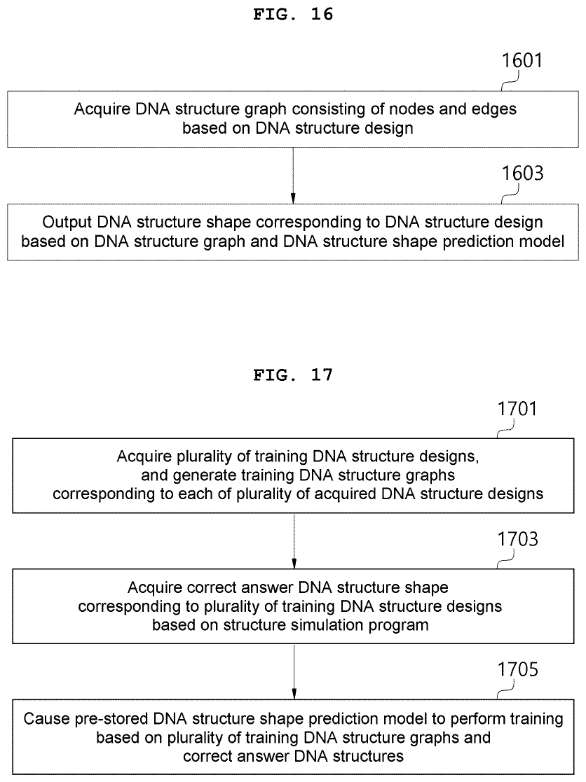

FIG. 16 is a flow chart illustrating the procedures of the operation for predicting the structure shape using the shape prediction model in the device according to an embodiment of the present invention.

In step 1601, the graph generation unit 203 may acquire a DNA structure graph consisting of nodes and edges based on the DNA structure design.

Here, the DNA structure design may be a DNA structure design acquired through the design acquisition unit 201. In addition, the structure design may be a structure design for the supramolecular assembly.

The graph generation unit 203 may model the nodes and edges based on base pairs included in the DNA structure design and a binding relationship between the base pairs, and generate a DNA structure graph including the nodes and edges.

Here, the node may include information on the position and orientation of the base pairs, and the edge may include a structural edge representing a covalent bond between the base pairs and an electromagnetic edge representing an electrostatic interaction due to the negative charge of DNA.

In step 1603, the structure prediction unit 209 may output the DNA structure shape corresponding to the DNA structure design based on the DNA structure graph and a pre-trained DNA structure shape prediction model (the shape prediction model).

Here, the DNA structure shape prediction model may consist of an ensemble model by including two or more DNA structure shape prediction models.

To describe in more detail, the ensemble model may include: a first DNA structure shape prediction model trained using the plurality of training DNA structure designs as one class; and two or more second DNA structure shape prediction models trained using the first DNA structure shape prediction model for each of two or more detailed classes. Here, each of the two or more detailed classes may have the plurality of training DNA structure designs classified and included according to the two or more detailed classes.

According to one embodiment, the detailed classes may be classified based on the structure of the DNA structures. For example, the detailed class may be classified into classes including at least one structure of the block structure, curved structure, twisted structure, hinged structure, two-dimensional wireframe structure, and three-dimensional wireframe structure.

The structure prediction unit 209 may acquire two or more preliminary DNA structure shapes with respect to an input of the DNA structure graph for each of two or more DNA structure shape prediction models included in the DNA structure shape prediction model. The structure prediction unit 209 may determine a preliminary DNA structure shape with the lowest energy among the two or more preliminary DNA structure shapes as the DNA structure shape.

FIG. 17 is a flow chart illustrating procedures of an operation for training the shape prediction model configured to predict the structure shape in the device according to an embodiment of the present invention.

In step 1701, the design acquisition unit 201 may acquire the plurality of training DNA structure designs, and the graph generation unit 203 may generate a training DNA structure graphs corresponding to each of the plurality of acquired DNA structure designs.

Here, the design acquisition unit 201 may acquire training augmentation designs by performing data augmentation based on insertion of at least one base pair or deletion of at least one base pair on at least some of the plurality of acquired training DNA structure designs.

Thereafter, the graph generation unit 203 may generate training augmentation structure graphs corresponding to each of the training augmentation structure designs.

In step 1703, the correct answer shape acquisition unit 205 may acquire the correct answer DNA structure shape corresponding to the plurality of training DNA structure designs based on the structure simulation program (e.g., SNUIPI).

In this case, when the structure simulation program is provided outside of the device 100 (e.g., a server), the correct answer shape acquisition unit 205 may receive the correct answer DNA structure shape corresponding to the plurality of training DNA structure designs from the server.

In step 1705, the learning processing unit 207 may cause a pre-stored DNA structure shape prediction model to perform training based on the plurality of training DNA structure graphs and the correct answer DNA structure.

Here, when the training augmentation structure graph is generated as described above, the learning processing unit 207 may cause the pre-stored DNA structure shape prediction model to perform training based on the plurality of training DNA structure graphs, the training augmentation structure graph, and the correct answer DNA structure.

The learning processing unit 207 may cause the pre-stored DNA structure shape prediction model to perform training by applying different loss functions to learning of the training DNA structure graphs and learning of the training augmentation structure graphs.

For example, the learning processing unit 207 may cause the DNA structure shape prediction model to perform training by applying a loss function based on at least some of the data-driven loss function and the physics-informed loss function to learning of the training DNA structure graph or learning of the training augmentation structure graph.

To describe in more detail, the learning processing unit 207 causes the pre-stored DNA structure shape prediction model to perform training on the training DNA structure graphs by applying a first loss function based on the data-driven loss and the physics-informed loss.

In addition, the learning processing unit 207 may cause the pre-stored DNA structure shape prediction model to perform training on the training augmentation structure graphs by applying at least some of the first loss function and a second loss function based on the physics-informed loss.

According to various embodiments of the present invention, the DNA structure shape prediction model trained by the hybrid training algorithm based on the loss function according to data-driven loss and physics-informed loss and the device including the same may be provided, thus to predict quickly and accurately the shape of the DNA origami structure.

According to various embodiments of the present invention, the ensemble model including the plurality of shape prediction models classified and trained according to the DNA structure and the device including the same may be provided, thus to predict the shape of the monomeric DNA origami structure with high precision in almost real time.

According to various embodiments of the present invention, the ensemble model based on the hybrid training algorithm in which the data-driven training method and the physics-informed training method are combined and the device including the same may be provided, thus to predict quickly and accurately a DNA structure having a complex shape in almost real time, and predict accurately the shapes of bio, nano, and macro structures at various scales, where they lack data.

In the present invention, any function performed by the device 100 has been described through various embodiments, but if a component that performs the corresponding function is not described as a component of the device 100, it should be understood that the device 100 includes a commonly known component that performs the function and/or is connected thereto.

As described above, although the embodiments have been described with reference to the limited drawings, it will be apparent to those skilled in the art that various modifications and alternations may be applied thereto based on the various embodiments.

For example, adequate effects may be achieved even if the foregoing processes and methods are carried out in different order than those described above, and/or the above-described elements, such as systems, structures, devices, or circuits, are combined or coupled in different forms and modes than those described above, or substituted or switched with other components or equivalents.

In particular, when describing with reference to the flowchart, it has been described that a plurality of steps are configured and the steps are sequentially executed in a designated order, but it is not necessarily limited to the designated order.

In other words, executing by changing or deleting at least some of the steps described in the flowchart or adding at least one step is applicable as an embodiment, and executing one or more steps in parallel may also be applicable as an embodiment. That is, it is not limited to that the steps are necessarily operated in a time-series order, and should be included in various embodiments of the present disclosure.

Therefore, other implements, other embodiments, and equivalents to claims are within the scope of claims to be described below.

1. A method for predicting a shape of a DNA structure based on deep learning, the method comprising:
acquiring a DNA structure graph having nodes and edges based on a DNA structure design; and
outputting a DNA structure shape corresponding to the DNA structure design based on the DNA structure graph and a DNA structure shape prediction model.

2. The method according to claim 1, wherein the DNA structure shape prediction model includes two or more DNA structure shape prediction models, and
the outputting of the DNA structure shape comprises:
acquiring two or more preliminary DNA structure shapes with respect to an input of the DNA structure graph for each of the two or more DNA structure shape prediction models; and
outputting a preliminary DNA structure shape with the lowest energy of the two or more preliminary DNA structure shapes as the DNA structure shape.

3. The method according to claim 1, wherein the DNA structure shape prediction model is generated by a process comprising:
acquiring a plurality of training DNA structure designs, and generating training DNA structure graphs corresponding to each of the acquired plurality of DNA structure designs;
acquiring a correct answer DNA structure shape corresponding to the plurality of training DNA structure designs based on a structural simulation program; and
causing a pre-stored DNA structure shape prediction model to perform training based on the plurality of training DNA structure graphs and the correct answer DNA structure.

4. The method according to claim 3, wherein the acquiring the plurality of training DNA structure designs comprises acquiring training augmentation designs by performing data augmentation based on insertion of at least one base pair or deletion of at least one base pair on at least some of the plurality of acquired training DNA structure designs,
the generating of the training DNA structure graphs comprises generating training augmentation structure graphs corresponding to each of the training augmentation structure designs, and
the causing of the pre-stored DNA structure shape prediction model comprises causing the pre-stored DNA structure shape prediction model to perform training based on the plurality of training DNA structure graphs, the training augmentation structure graphs, and the correct answer DNA structures.

5. The method according to claim 4, wherein the causing of the pre-stored DNA structure shape prediction model comprises causing the pre-stored DNA structure shape prediction model to perform training by applying different loss functions to learning of the training DNA structure graphs and learning of the training augmentation structure graphs.

6. The method according to claim 4, wherein the causing of the pre-stored DNA structure shape prediction model comprises causing the pre-stored DNA structure shape prediction model to perform training on the training DNA structure graphs by applying a first loss function based on a data-driven loss and a physics-informed loss.

7. The method according to claim 4, wherein the causing of the pre-stored DNA structure shape prediction model comprises causing the pre-stored DNA structure shape prediction model to perform training on the training augmentation structure graphs by applying a second loss function based on a physics-informed loss.

8. The method according to claim 3, wherein the DNA structure shape prediction model comprises:
a first DNA structure shape prediction model trained using the plurality of training DNA structure designs as one class; and two or more second DNA structure shape prediction models trained using the first DNA structure shape prediction model for each of two or more detailed classes; and
each of the two or more detailed classes has the plurality of training DNA structure designs classified and included according to the two or more detailed classes.

9. A device for predicting a shape of a DNA structure based on deep learning, the device comprising:
a storage unit in which a DNA structure shape prediction model is stored;
a graph generation unit configured to acquire a DNA structure graph consisting of nodes and edges based on a DNA structure design; and
a structure prediction unit configured to output a DNA structure shape corresponding to the DNA structure design based on the DNA structure graph and a pre-trained DNA structure shape prediction model.

10. The device according to claim 9, wherein the DNA structure shape prediction model includes two or more DNA structure shape prediction models, and
the structure prediction unit is configured to:
acquire two or more preliminary DNA structure shapes with respect to an input of the DNA structure graph for each of the two or more DNA structure shape prediction models included in the DNA structure shape prediction model included in the DNA structure shape prediction models; and
determine a preliminary DNA structure shape with the lowest energy of the two or more preliminary DNA structure shapes as the DNA structure shape.

11. The device according to claim 10, further comprising:
a design acquisition unit;
a correct answer shape acquisition unit; and
a learning processing unit,
wherein the DNA structure shape prediction model is generated by a processes comprising:
acquiring, by the design acquisition unit, a plurality of training DNA structure designs;
generating, by the graph acquisition unit, training DNA structure graphs corresponding to each of the acquired plurality of DNA structure designs;
acquiring, by the correct answer shape acquisition unit, a correct answer DNA structure shape corresponding to the plurality of training DNA structure designs based on a structural simulation program; and
causing, by the learning processing unit, the pre-stored DNA structure shape prediction model to perform training based on the plurality of training DNA structure graphs and the correct answer DNA structure.

12. The device according to claim 11, wherein the design acquisition unit is configured to acquire training augmentation designs by performing data augmentation based on insertion of at least one base pair or deletion of at least one base pair on at least some of the plurality of acquired training DNA structure designs,
the graph acquisition unit is configured to generate training augmentation structure graphs corresponding to each of the training augmentation structure designs, and
the learning processing unit is configured to cause the pre-stored DNA structure shape prediction model to perform training based on the plurality of training DNA structure graphs, the training augmentation structure graphs, and the correct answer DNA structure.

13. The device according to claim 12, wherein the learning processing unit is configured to cause the pre-stored DNA structure shape prediction model to perform training by applying different loss functions to learning of the training DNA structure graphs and learning of the training augmentation structure graphs.

14. The device according to claim 12, wherein the learning processing unit is configured to cause the pre-stored DNA structure shape prediction model to perform training on the training DNA structure graphs by applying a first loss function based on a data-driven loss and a physics-informed loss.

15. The device according to claim 12, wherein the learning processing unit is configured to cause the pre-stored DNA structure shape prediction model to perform training on the training augmentation structure graphs by applying a second loss function based on a physics-informed loss.

16. The device according to claim 12, wherein the learning processing unit is configured to generate the DNA structure shape prediction model by including:
a first DNA structure shape prediction model trained using the plurality of training DNA structure designs as one class; and
two or more second DNA structure shape prediction models trained using the first DNA structure shape prediction model for each of two or more detailed classes, and
each of the two or more detailed classes has the plurality of training DNA structure designs classified and included according to the two or more detailed classes.
